(19) United States
(12) Patent Application Publication
(10) Pub. No.: US 2015/0065579 A1
(43) Pub. Date: Mar. 5, 2015

(54) POLYCATIONIC POLYHYDROXYL COMPOUNDS

(71) Applicant: DOW GLOBAL TECHNOLOGIES LLC, Midland, MI (US)

(72) Inventor: Deavenport L. Joseph, Lake Jackson, TX (US)

(21) Appl. No.: 14/389,511

(22) PCT Filed: Mar. 26, 2013
(86) PCT No.: PCT/US2013/033777

§ 371 Date: Sep. 30, 2014

Related U.S. Application Data
(60) Provisional application No. 61/618,009, filed on Mar. 30, 2012.

(51) Int. Cl. C07C 215/40; A61Q 17/04; A61Q 19/10; A61K 8/41; A61Q 5/06

(52) U.S. Cl. CPC C07C 215/40; A61K 8/416; A61Q 5/06; A61Q 19/10; A61Q 17/04; A61K 2800/10
USPC 514/642; 564/292

(57) ABSTRACT

Described are novel polycationic polyhydroxyl compounds and their uses in personal care compositions.

### FIELD

The present invention relates to novel polycationic polyhydroxyl compounds and their uses in personal care compositions.

### BACKGROUND

Polyhydroxyl compounds, or polyols, have a number of uses, from raw materials used in the manufacture of urethane foams to humectants for personal care products like shaving foams, skin and sun care lotions and sprays, hair conditioning formulations, hair color formulations, make-up formulations, anti-wrinkle formulations, formulations that promote hair volume or shine, hair styling products such as those in the mousse, lotion, wax, aerosol, or gel form for fixing a hair style, skin and facial cleansing liquids, bars, and foams, hair removers, sunscreens, and shampoos.

Quaternary ammonium compounds are also useful in a number of applications, such as for disinfectants, surfactants, fabric softeners, and conditioners in shampoos.

Despite the number of available conventional compounds, there is a strong need for novel compounds with properties to differentiate performance or offer synergistic effects in areas of interest, particularly in personal care compositions.

### DETAILED DESCRIPTION

In one embodiment, the present invention provides compounds, including salts, of the Formula (I):

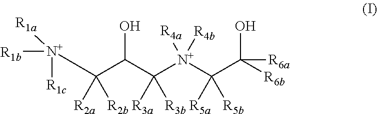

wherein:

R₁ₐ, R_{1b}, R_{1c}, R₂ₐ, R_{2b}, R₃ₐ, R_{3b}, R₅ₐ, R_{5b}, R₆ₐ, and R_{6b}, are, independently, H,

optionally substituted C1-C6 alkyl, or R₂ₐ and R_{2b}, R₃ₐ and R_{3b}, or R₅ₐ and R_{5b}, may cooperate to form a cycloalkyl; and

R₄ₐ and R_{4b} are, independently, optionally substituted C1-C6 alkyl,

provided that at least one of R₄ₐ, R_{4b}, R₅ₐ, R_{5b}, R₆ₐ, and R_{6b} is C1-C6 alkyl substituted with at least one hydroxy group or amino group.

The term “optionally substituted” as used herein means that the groups in question are either unsubstituted or substituted with one or more groups, radicals or moieties, selected from halogen, hydroxy, amino or carboxy. “Amino” is intended to include amino further substituted with C1-C3alkyl, preferably trimethylamino (—N⁺(CH₃)₃). When the groups in question are substituted with more than one substituent, the substituents may be the same or different. In one embodiment, the optional substituent is selected to produce a cosmetically acceptable compound. “Cosmetically acceptable” refers to ingredients typically used in personal care compositions, and is intended to underscore that materials that are toxic, irritating, or unpleasant smelling when present in the amounts typically found in personal care compositions are not contemplated as part of the present invention. In one embodiment, the optional substituent is one or more hydroxy groups.

“Alkyl” means a saturated monovalent linear or branched aliphatic hydrocarbon radical. Representative examples include, but are not limited to methyl, ethyl, propyl, isopropyl, butyl, isobutyl, tert-butyl, sec-butyl, pentyl, isopentyl, neopentyl, hexyl, isohexyl, and the like.

The term “cycloalkyl” denotes a saturated monocyclic or bicyclic cycloalkyl group. Examples of cycloalkyl groups include cyclopropyl, cyclobutyl, cyclopentyl, cyclohexyl, cycloheptyl, cyclooctyl, and the like. In one embodiment, the cycloalkyl is cyclohexyl or cyclopentyl.

Salts means that a counter-ion is present, preferably halogen, more preferably Cl⁻.

In one embodiment, R₁ₐ, R_{1b}, R_{1c}, are each methyl.

In one embodiment, R₂ₐ, R_{2b} R₃ₐ, and R_{3b} are each H.

In one embodiment, R₄ₐ and R_{4b} are each —CH₃. In an alternative embodiment, R₄ₐ and R_{4b} are each —CH₂CH(OH)CH₂OH. In yet another alternative embodiment, R₄ₐ and R_{4b} are each —CH₂CH₂OH. In yet another alternative embodiment, R₄ₐ is —(CH₂)₃CH₃ and R_{4b} is —CH₂CH₂OH. In yet another alternative embodiment, R₄ₐ is —CH₃ and R_{4b} is —CH₂CH(OH)CH₂OH.

In one embodiment, the present invention provides compounds, including salts, of the Formula (II):

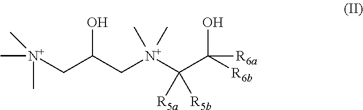

wherein:

R₅ₐ, R_{5b}, R₆ₐ, and R_{6b}, are, independently, H, optionally substituted C1-C6 alkyl, or R₅ₐ and R_{5b} may cooperate to form a cycloalkyl, provided that at least one of R₅ₐ, R_{5b}, R₆ₐ, and R_{6b} is C1-C6 alkyl substituted with at least one hydroxy group or amino group.

In one embodiment, R₅ₐ and R_{5b} are each H, and at least one of R₆ₐ or R_{6b} is alkyl, preferably C1-C3alkyl, substituted with a trimethylamino group (—N⁺(CH₃)₃).

In one embodiment, R₅ₐ and R_{5b} are each C1-C3alkyl, wherein at least one of R₅ₐ and R_{5b} are preferably C1-C3alkyl substituted with a hydroxy group, and R₆ₐ and R_{6b} are each H.

Non-limiting examples of compounds of Formula II include the reaction products of 2,3-epoxypropyltrimethylammonium chloride and 2-(dimethylamino)-2-methylpropane-1,3-diol, 2,3-epoxypropyltrimethylammonium chloride and 2-(dimethylamino)propan-1,3-diol, 2,3-epoxypropyltrimethylammonium chloride and 2-(dimethylamino)-2-(hydroxymethyl)propane-1,3-diol.

It is understood that many other embodiments where R₄ₐ and R_{4b} are not both methyl are contemplated, for example, the reaction products of 2,3-epoxypropyltrimethylammonium chloride and 2-(diethylamino)-2-(hydroxymethyl)propane-1,3-diol, 2,3-epoxypropyltrimethylammonium chloride and 2-(ethyl(hydroxymethyl)amino)-2-(hydroxymethyl)propane-1,3-diol, 2,3-epoxypropyltrimethylammonium chloride and 2-(bis(hydroxymethyl)amino)-2-(hydroxymethyl)propane-1,3-diol, 2,3-epoxypropyltrimethylammonium chloride and 2-(diethylamino)propane-1,3-diol, 2,3-epoxypropyltrimethylammonium chloride and 2-(ethyl(hydroxymethyl)amino)propane-1,3-diol, 2,3-epoxypropyltrimethylammonium chloride and 2-(bis(hydroxymethyl)amino)propane-1,3-diol, 2,3-epoxypropyltrimethylammonium chloride and 2-(diethylamino)-2-methylproane-1,3-diol, 2,3-epoxypropyltrimethylammonium chloride and 2-(bis(2-hydroxyethyl)amino)-2-methylpropane-1,3-diol, 2,3-epoxypropyltrimethylammonium chloride and 2-(ethyl(hydroxymethyl)amino)-2-methylproane-1,3-diol, 2,3-epoxypropyltrimethylammonium chloride and 2-(bis(hydroxymethyl)amino)-2-methylpropane-1,3-diol, 2,3-epoxypropyltrimethylammonium chloride and 2-(dipropylamino)-2-methylpropane-1,3-diol, 2,3-epoxypropyltrimethylammonium chloride and 3,3′-((1,3-dihydroxy-2-(hydroxymethyl)propan-2-yl)azanediyl)bis(propane-1,3-diol), 2,3-epoxypropyltrimethylammonium chloride and 3,3′-((1,3-dihydroxy-2-methylpropan-2-yl)azanediyl)bis(propane-1,2-diol), 2,3-epoxypropyltrimethylammonium chloride and 2-(bis(hydroxymethyl)amino)propane-1,3-diol, 2,3-epoxypropyltrimethylammonium chloride and 3,3′-((1,3-dihydroxypropan-2-yl)azanediyl)bis(propane-1,3-diol.

In one embodiment, the present invention provides methods for providing humectancy in a personal care composition, comprising including the compound of Formula I into the personal care composition. The ingredients used, and their proportions and manner of addition, are familiar to those versed in conventional personal care compositions, including, optionally, cosmetically acceptable emollients and moisturizers such as for example glycerin, mineral oil/petrolatum, propylene glycol, sorbitol, polyethylene glycol, conditioners such as for example quaternary ammonium conditioners, polyquaternium conditioners such as polyquaternium-10, 67, polyolefins, guar hydroxypropyltrimonium chloride, oils such as triglyceride oils, silicone oils, hydrocarbon oils, waxes, sunscreen UV filters, surfactants, emulsifiers, preservatives, rheology modifiers, texture modifiers such as silicones or silicone elastomers, polyglycols, HEC, HPMC, colorants or pigments for the hair or skin, preservatives, pH adjustors, propellants, reducing agents, fragrances, foaming or de-foaming agents, tanning agents, depilatory agents, astringents, antiseptics, deodorants, antiperspirants, insect repellants, bleaches, lighteners, anti-dandruff agents, adhesives, polishes, strengtheners, fillers, barrier materials, hair fixative film forming polymers such as, for example those under the INCI name of acrylates/hydroxyesters acrylates copolymer or styrene/acrylates copolymer or biocides.

In one embodiment, the present invention provides hair care compositions containing the compound of Formula I or Formula II.

In one embodiment, the present invention provides skin care compositions containing the compound of Formula I or Formula II.

In one embodiment, the present invention provides sun care compositions containing the compound of Formula I or Formula II.

### EXAMPLES

The following examples are for illustrative purposes only and are not intended to limit the scope of the present invention.

### Example 1

### Reaction of bis(3-chloro-2-hydroxypropyl)dimethylammonium Chloride and Trimethylamine to Afford 2-hydroxy-N¹-(2-hydroxy-3-(trimethylammonio)propyl)-N¹, N¹, N³, N³, N³-pentamethylpropane-1,3-diaminium Chloride

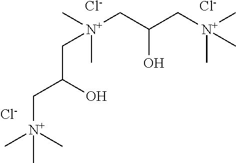

1.6253 g of bis(3-chloro-2-hydroxypropyl)dimethylammonium chloride (42.5% solution in water) was added to a glass vial. 1.1760 g trimethylamine (43.6% solution in water) was added to the vial and the solution was stirred at ambient temperature for 16 hours. The solution was heated to 60° C. for 4 hours.

¹³C NMR spectra acquired from a Bruker 300 MHz spectrometer (samples prepared as ˜30 wt % in D₂O) confirmed the title compound: DEPT NMR (250 MHz, D2O) (52.9, 54.6, 62.0, 66.5, 67.7)

### Example 2

### Reaction of 2,3-epoxypropyltrimethylammonium Chloride and Dimethylaminoethyl-propanediol to Afford 2-hydroxy-N¹-(1-hydroxy-2-(hydroxymethyl)butan-2-yl)-N¹, N¹, N³, N³, N³ -pentamethylpropane-1,3-diaminium Chloride

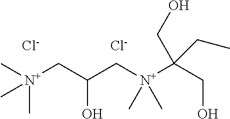 [SMILES: CCC(CO)(CO)[N+](C)(C)CC(O)C[N+](C)(C)C ; [Cl-] ; [Cl-]]

A 250 mL round bottom, jacketed, flask was equipped with a magnetic stirbar and placed on a stir plate. The temperature was set to 13° C. and monitored with a thermocouple. 3-chloro-2-hydroxypropyltrimethylammonium chloride (Quat 188, 69%, commercially available from The Dow Chemical Company), 51.63 g (0.19 mol), was added to the reactor flask. Next, 15.87 g sodium hydroxide (49.8%, 0.20 mol), was added dropwise over about 10 minutes while maintaining a temperature of about 15° C.

The solution was allowed to stir for one hour at 13° C. The temperature was then set to 4° C. and 23.26 g of dimethylaminoethylpropanediol (0.16 mol) was added. The temperature was set to 45° C. and the reaction mixture was allowed to stir for three hours whereupon the pH was decreased to 7 by adding 33.47 g hydrochloric acid (13%). NMR confirmed reaction completion.

¹³C NMR spectra acquired from a Bruker 300 MHz spectrometer (samples prepared as ˜30 wt % in D2O) confirmed the title compound: DEPT NMR (250 MHz, D2O) 9.9, 23.1, 41.1, (56.7, 57.0), 62.3, 64.7, 66.1, 70.6.

### Example 3

Compounds prepared substantially according to the Examples 1-2 are made and formulated into personal care compositions having otherwise conventional ingredients. The compositions are evaluated by trained panelists, with each panelist being asked to compare the inventive compositions to a conventional composition.

For hair care compositions, wet and dry feel preference and wet and dry combability is measured by asking the panelists to feel and comb two hair tresses of European virgin brown hair, commercially available from International Hair Importers and Products Inc. NY (USA), one hair tress treated with an inventive composition, the other hair tress treated with a conventional composition. Each panelist is asked to compare the tresses and state which tress is smoother to comb/feel. The answer “same” is not allowed. The reported number is the percent of panelists preferring one over the other.

For skin care compositions, panelists apply a sample (one inventive composition, one conventional composition) to a designated area on their right or left forearm. Initially, each sample is evaluated for ease of application, play time, evenness of deposit, coverage, speed of adsorbtion, shine, matte, skin moistness, heaviness, amount of grease, amount of tack, quickness of drying, overall skin feel, and overall appearance. After a designated time, each sample is again evaluated, this time for coverage, evenness of coverage, shine, matte, skin moistness, heaviness, and overall appearance.

1. A salt comprising an ion of the Formula (I): 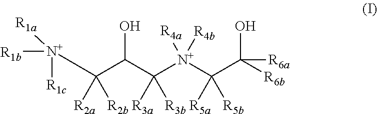
wherein:
R₁ₐ, R_{1b}, R_{1c}, R₂ₐ, R_{2b}, R₃ₐ, R_{3b}, R₅ₐ, R_{5b}, R₆ₐ, and R_{6b}, are, independently, H,
optionally substituted C1-C6 alkyl, or R₂ₐ and R_{2b}, R₃ₐ and R_{3b}, or R₅ₐ and R_{5b}, may cooperate to form a cycloalkyl; and
R₄ₐ and R_{4b} are, independently, optionally substituted C1-C6 alkyl, provided that at least one of R₄ₐ, R_{4b}, R₅ₐ, R_{5b}, R₆ₐ, and R_{6b} is C1-C6 alkyl substituted with at least one hydroxy group or amino group.

2. The salt of claim 1, wherein R₁ₐ, R_{1b}, and R_{1c} are each methyl.

3. The salt of claim 1, wherein R₄ₐ and R_{4b} are each —CH₃.

4. The salt of claim 1, wherein R₄ₐ and R_{4b} are each —CH₂CH(OH)CH₂OH.

5. The salt of claim 1, wherein R₄ₐ and R_{4b} are each —CH₂CH₂OH.

6. The salt of claim 1, wherein R₄ₐ is —(CH₂)₃CH₃ and R_{4b} is —CH₂CH₂OH.

7. The salt of claim 1, having the Formula (II): 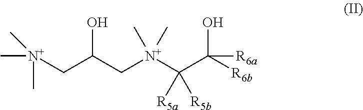
wherein:
R₅ₐ, R_{5b}, R₆ₐ, and R_{6b}, are, independently, H, optionally substituted C1-C6 alkyl, or R₅ₐ and R_{5b} may cooperate to form a cycloalkyl, provided that at least one of R₅ₐ, R_{5b}, R₆ₐ, and R_{6b} is C1-C6 alkyl substituted with at least one hydroxy group or amino group.

8. The salt of claim 7, wherein R₅ₐ and R_{5b} are each H, R₆ₐ is H, and R_{6b} is
—CH₂N⁺(CH₃)₃).

9. The salt of claim 7, wherein R₅ₐ is ethyl, R_{5b} is —CH₂OH, and R₆ₐ and R_{6b} are each H.

10. A method for providing humectancy in a personal care composition, comprising adding of a salt of claim 1 into the personal care composition.

11. A hair care composition containing a salt of claim 1.

12. A skin care composition containing a salt of claim 1.

13. A sun care composition containing a salt of claim 1.
